(19) United States
(12) Patent Application Publication
(10) Pub. No.: US 2004/0258647 A1
(43) Pub. Date: Dec. 23, 2004

(54) Active ingredient-containing cosmetic cleansing emulsions

(75) Inventors: Stephan Ruppert, Hamburg (DE); Michaela Kohut, Hamburg (DE)

Correspondence Address: GREENBLUM & BERNSTEIN, P.L.C., 1950 ROLAND CLARKE PLACE, RESTON, VA, 20191, US

(73) Assignee: BEIERSDORF AG, Hamburg (DE)

(21) Appl. No.: 10/830,000

(22) Filed: Apr. 23, 2004

Related U.S. Application Data
(63) Continuation of application No. PCT/EP02/11750, filed on Oct. 21, 2002.

(30) Foreign Application Priority Data
Oct. 26, 2001 (DE) 101 53 023.4

(51) Int. Cl. A61K 7/06; A61K 7/11

(52) U.S. Cl. 424/70.16

(57) ABSTRACT

Cosmetic or dermatological cleansing emulsions, characterized in that, based on the total weight of the preparations, they contain

1 to 30% by weight of one or more detergent surfactants, chosen from the group consisting of the surfactants which have an HLB of more than 15,

35 to 50% by weight of one or more oil components,

0.001 to 30% by weight of one or more active ingredients,

0.2 to 5% by weight of one or more polyacrylates, chosen from the group which is formed from anionic homo- and/or copolymers of acrylic acid and/or alkylated acrylic acid derivatives, and their esters and

5 to 60% by weight of water.

### CROSS-REFERENCE TO RELATED APPLICATIONS

The present application is a continuation of International Application No. PCT/EP02/11750, filed Oct. 21, 2002, the entire disclosure whereof is expressly incorporated by reference herein, which claims priority under 35 U.S.C. § 119 of German Patent Application No. 101 53 023.4, filed Oct. 26, 2001.

The present invention relates to active ingredient-containing cosmetic cleansing emulsions, in particular those which contain no emulsifier in the conventional sense.

The production of cosmetic cleansing agents has been showing a rising trend for years. This is especially to be attributed to the increasing health consciousness and need for hygiene of the consumers.

Cleansing means the removal of (environmental) dirt and thus brings about an increase in psychological and physical wellbeing. The cleansing of the surface of skin and hair is a very complex process dependent on many parameters. On the one hand, substances originating from outside such as, for example, hydrocarbons or inorganic pigments from all sorts of different fields and residues of cosmetics or alternatively undesirable microorganisms should be removed as completely as possible. On the other hand, endogenous secretions such as perspiration, sebum, skin scales and dandruff are to be washed off without far-reaching interventions into the physiological equilibrium.

The demands on the properties of cosmetic cleansing preparations have varied greatly in recent years. Earlier, effects such as cleansing and foaming were foremost in the wishes of consumers. At present, the ecological, economical and in particular dermatological properties of the products are of prime importance, although the foaming power still plays a crucial role, for example as an indicator in order to remove residual amounts of surfactants from skin and hair after cleansing or in order to avoid overdosages during application. However, in the case of cosmetic products—in contrast to most industrial cleansing agents—the skin and mucous membrane compatibility are absolutely foremost; the products should be “mild”.

Cosmetic or dermatological cleansing preparations are “rinse off” preparations which are rinsed off the skin after application. They are as a rule applied to the parts of the body to be cleansed in the form of a foam with water. Detergent surfactants are the basis of all cosmetic or dermatological cleansing preparations. Surfactants are amphiphilic substances which can dissolve organic, nonpolar substances in water. They are distinguished by an ambivalent behavior to water and lipids: The surfactant molecule contains at least one hydrophilic and one lipophilic group each, which make possible the accumulation on the interface between these two classes of substance. In this way, surfactants provide for a reduction in the surface tension of the water, the wetting of the skin, the facilitation of dirt removal and dissolution, easy rinsing off and —if desired—also for foam regulation. The basis for dirt removal from lipid-containing soilings is thus afforded.

The hydrophilic portions of a surfactant molecule are usually polar functional groups, for example —COO⁻, —OSO₃²⁻, —SO₃⁻, while the hydrophobic parts as a rule are nonpolar hydrocarbon radicals. Surfactants are in general classified according to the nature and charge of the hydrophilic moiety. Four groups can be be differentiated here:

anionic surfactants,

cationic surfactants,

amphoteric surfactants and

nonionic surfactants.

As functional groups, anionic surfactants as a rule contain carboxylate, sulfate or sulfonate groups. In aqueous solution, they form negatively charged organic ions in the acidic or neutral medium. Cationic surfactants are almost exclusively characterized by the presence of a quaternary ammonium group. In aqueous solution, they form positively charged organic ions in the acidic or neutral medium. Amphoteric surfactants contain both anionic and cationic groups and accordingly behave in aqueous solution, depending on the pH, as anionic or cationic surfactants. In the strongly acidic medium they have a positive charge and in alkaline medium a negative charge. In the neutral pH region, however, they are zwitterionic, as the following example is intended to illustrate:

RNH₂⁺CH₂CH₂COOH X⁻ (at pH=2) X⁼ any desired anion, e.g. Cl⁻

RNH₂⁺CH₂CH₂COO⁻ (at pH=7)

RNHCH₂CH₂COO⁻ B³⁰ (at pH=12) B⁺=any desired cation, e.g. Na⁺

Polyether chains are typical of nonionic surfactants. Nonionic surfactants do not form any ions in aqueous medium.

The detergent surfactants in cosmetic and dermatological cleansing agents are subject to a very critical assessment with respect to their dermatological and ecological behavior. The latter is in particular of importance, since they are used in considerable amount and, after use, pass into the waste water as intended.

Starting from the already described central importance of the detergent surfactants for the cleansing process, their behavior on the human skin is of a very great importance.

Even during cleansing of the skin with the aid of water—without the addition of surfactants—a swelling of the horny layer of the skin first occurs. The degree of this swelling depends, inter alia, on the duration of the bath and its temperature. At the same time, water-soluble substances are washed off or out, such as, for example, water-soluble dirt constituents, but also the skin's own substances, which are responsible for the water-binding power of the horny layer. By means of the skin's own surface-active substances, skin fats are moreover also dissolved to a certain extent and washed out. After initial swelling, this causes a subsequent drying out of the skin.

It is understandable that detergent surfactants which are intended to cleanse the skin and hair of fatty and water-soluble dirt constituents also have a defatting action on the normal skin lipids. In any cleansing of the skin, to a differing extent, intercorneocytic lipids and sebum constituents are also removed. This means that the natural water/lipid coat of the skin is more or less destroyed in any washing process. This can lead, particularly in the case of extreme defatting, to a short-term change in the barrier function of the skin, where, of course, the particular condition of the region of the skin treated is of considerable influence on the changes shown. For example, the skin thickness, the number of sebaceous and sweat glands and the sensitivity associated therewith can vary considerably.

In principle, it is accordingly regarded as a requirement of detergent surfactants that they are as biologically inactive as possible in order to avoid undesired side effects. They should display their cleansing action with optimum mildness, best skin compatibility and low defatting.

There has in addition, however, also not been a lack of attempts to find suitable cleansing preparations which regenerate or “refat” the skin at the same time with good cleansing power. However, the performance achieved often remains below that expected, so that the user as a rule has to resort to separate care products, which are applied to the skin after cleansing and remain on this (“leave-on” products).

These products as a rule also contain a number of active ingredients which care for and regenerate the skin. They increase the barrier properties of the skin and decrease and prevent premature skin ageing (e.g. wrinkles and folds). It is their object to help the skin to a smooth and healthy youthful appearance.

Another object of the active ingredients is the acceleration of skin regeneration, which leads to a more rapid restoration of the natural equilibrium of the skin after washing with skin-irritating surfactants. A further object of the active ingredients is the replacement of lipids, humectant factors, vitamins and other skin constituents washed out during the washing process. Further, the active ingredients (UV filters) can serve for the protection of the skin from the harmful UV radiation of sunlight.

As a rule, cosmetic or dermatological cleansing preparations are very well tailored to an assumed application spectrum, since for a defined, mild cleansing action, the mechanical parameters—such as, for example, the time factor—which are different depending on the application are in particular also of considerable importance: This, for example, becomes plain if the different application (contact) times of a foam bath in comparison with brief handwashing are made clear.

Cosmetic cleansers usually contain mixtures of surfactants of various types. The choice is orientated primarily to the skin compatibility and the desired cosmetic performance of the surfactants. In addition, foaming power, formulability and a favorable performance/cost ratio play an important role.

Liquid soaps or washing lotions are not only used for cleansing the hands, but as a rule also for the whole body, including the face. They are accordingly also suitable for use as a shower preparation. In the development of these products, the dermatological requirements are foremost, since the skin comes into intensive contact with the concentrated surfactant solution. Particular value is therefore placed on the selection of mild surfactants in low concentration. Further criteria are furthermore a good foaming power and a pleasant, refreshing fragrance and the simultaneous care of the skin. Washing lotions and in particular shower baths as a rule have viscosities of approximately 3000 to 10,000 mPa·s, which on the one hand allow a good dispersibility of the product with rapid foaming, but at the same time on the other hand should be high enough in order to make possible perfect application by hand or flannel.

Liquid soaps or washing lotions are in general distinguished by a more or less high water content, but as a rule display no noticeable care action, since they only contain a low oil content.

Surfactant-containing shower preparations having a high oil content are a relatively new technical development. German laid-open application 44 24 210 in this connection describes cosmetic or dermatological shower preparations having a surfactant content of at most 55% by weight and an oil content of more than 45% by weight, the preparations essentially being water-free. On account of the high oil content, these preparations have a regenerating action with respect to the general state of the skin. In this case, they at the same time have a good foam development and a high cleansing power.

WO 96/17591 further describes foaming liquid skin cleansing compositions which contain the following substances: 5 to 30% by weight of a moisture-donating active ingredient, which has a Vaughan solubility parameter (VSP) of 5 to 10, 0.3 to 5% by weight of a water-dispersible gel-forming polymer, 5 to 30% by weight of a synthetic surface-active substance, 0 to 15% by weight of a C₈ to C₁₄ fatty acid soap and water, where the preparations have a lipid deposition value (LDV) of at least 5 to 1000 and in which the synthetic surface-active substance and the soap have a common CMC equilibrium surface tension value of 15 to 50. However, this specification was unable to point the way to the present invention.

For the cleansing and simultaneous care of the skin, the prior art furthermore also knows emulsion-based cleansing products. These are formulated by stabilizing the emulsion with emulsifiers and subsequently tailoring a surfactant system. Emulsifiers also have an amphiphilic structure, and are thus comparable to the surfactants as far as structure is concerned. Emulsifiers make possible or facilitate the uniform dispersion of two or more phrases which are not miscible with one another and at the same time prevent their demixing. Since emulsions are in general destroyed by the addition of surfactants, the choice of the surfactant system is greatly restricted, and the cleansing preparations obtained are based on expensive and complicated recipes.

What then distinguishes detergent surfactants from emulsifiers?

At the end of the forties a system was developed which was intended to facilitate the choice of emulsifiers. Each emulsifier is ascribed an “HLB” (a dimensionless number between 0 and 20) which indicates whether a preferred water or oil solubility is present. Numbers below 9 indicate oil-soluble, hydrophobic emulsifiers, numbers over 11 water-soluble, hydrophilic emulsifiers. The HLB says something about the equilibrium between the size and strength of the hydrophilic and of the lipophilic groups of an emulsifier. From these considerations, it can be derived that the efficacy of an emulsifier can also be characterized by its HLB. The following list shows the relationship between HLB and possible application area:

| HLB | Application area |
|---|---|
| 0 to 3 | Antifoam |
| 3 to 8 | W/O emulsifier |
| 7 to 9 | Wetting agent |
| 8 to 18 | O/W emulsifier |
| 12 to 18 | Solubilizer |

The HLB of an emulsifier can also be composed of increments, where the HLB increments for the various hydrophilic and hydrophobic groups from which a molecule is composed can be taken from tables. In this way, HLBs can in principle also be determined for detergent surfactants, although the HLB system has originally been conceived only for emulsifiers. It is seen that detergent substances as a rule have HLBs which are markedly greater than 20.

It would be desirable to have available cleansing preparations on the basis of emulsions, which remedy the disadvantages of the prior art and which accordingly are based on simple and inexpensive recipes. The preparations should moreover have a high care action without the cleansing action taking second place to it. Furthermore, it should be possible to incorporate into the cleansing preparations active ingredients which prevent cosmetic and/or dermatological deficiencies of the skin such as, for example, the formation and deepening of wrinkles or decrease these.

The present invention further relates to detergent hair cosmetic preparations, in general described as shampoos. In particular, the present invention relates to hair cosmetic active ingredient combinations and preparations for the care of the hair and of the scalp. There is lack of shampoo formulations in the prior art which bestow care on damaged hair in a satisfactory manner.

It has surprisingly been shown that cosmetic or dermatological cleansing emulsions which comprise, based on the total weight of the preparations,

1 to 30% by weight of one or more detergent surfactants, chosen from the group consisting of the surfactants which have an HLB of more than 15,

35 to 50% by weight of one or more oil components,

0.001 to 30% by weight of one or more active ingredients,

0.2 to 5% by weight of one or more polyacrylates, chosen from the group which is formed from anionic homo- and/or copolymers of acrylic acid and/or alkylated acrylic acid derivatives, and their esters and

5 to 60% by weight of water,

may remedy the disadvantages of the prior art.

The cosmetic and/or dermatological cleansing emulsions within the meaning of the present invention are based on simple and inexpensive recipes. They simultaneously have a good foam development and a high cleansing power. On account of the high oil content, these preparations have a regenerating action with respect to the general state of the skin, decrease the feeling of dryness of the skin and make the skin supple.

The active ingredients incorporated into the cleansing emulsions according to the invention serve, inter alia, for the prophylaxis and/or for treatment of inflammatory skin conditions and/or skin protection in sensitive and dry skin (such as, for example, atopic eczema, seborrheic eczema, polymorphic photodermatosis, psoriasis, vitiligo, wound healing disorders, itching, sensitive or irritated skin, light-related skin damage and UV-induced immunosuppression, changes in desquamation, changes in normal fibroblast and keratinocyte proliferation, changes in normal fibroblast and keratinocyte differentiation, deficient sensitive or hypoactive skin conditions or deficient sensitive or hypoactive conditions of the skin appendages and for reducing the skin thickness).

The cleansing emulsions advantageously contain one or more detergent anionic, cationic, amphoteric and/or nonionic surfactants according to the invention. It is particularly advantageous to choose the detergent surfactant(s) according to the invention from the group consisting of the surfactants which have an HLB of more than 25; those which have an HLB of more than 35 are very particularly advantageous.

Particularly advantageous detergent anionic surfactants within the meaning of the present invention are

acylamino acids and their salts, such as

acylglutamates, in particular sodium acylglutamate

sarcosinates, for example myristoyl sarcosine, TEA lauroyl sarcosinate, sodium lauryl sarcosinate and sodium cocoyl sarcosinate,

sulfonic acids and their salts, such as

acyl isethionates, e.g. sodium/ammonium cocoyl isethionate,

sulphosuccinates, for example dioctyl sodium sulfosuccinate, disodium laureth sulfosuccinate, disodium lauryl sulfosuccinate and disodium undecyleneamido MEA sulfosuccinate

and sulfuric acid esters, such as

alkyl ether sulfate, for example sodium, ammonium, magnesium, MIPA, TIPA laureth sulfate, sodium myreth sulfate and sodium C₁₂₋₁₃ pareth sulfate,

alkyl sulfates, for example sodium, ammonium and TEA lauryl sulfate.

Particularly advantageous detergent cationic surfactants within the meaning of the present invention are quaternary surfactants. Quaternary surfactants contain at least one N atom which is covalently bonded to 4 alkyl or aryl groups. Benzalkonium chloride, alkylbetaine, alkylamidopropylbetaine and alkylamidopropylhydroxysultaine are advantageous.

Particularly advantageous detergent amphoteric surfactants within the meaning of the present invention are

Acyl-/dialkylethylenediamines, for example sodium acylamphoacetate, disodium acylamphodipropionate, disodium alkylamphodiacetate, sodium acyl-amphohydroxypropylsulfonate, disodium acylamphodiacetate and sodium acyl-amphopropionate,

Particularly advantageous detergent nonionic surfactants within the meaning of the present invention are

alkanolamides, such as cocamides MEA/DEA/MIPA,

esters which are formed by esterification of carboxylic acids with ethylene oxide, glycerol, sorbitan or other alcohols,

ethers, for example ethoxylated alcohols, ethoxylated lanolin, ethoxylated polysiloxanes, propoxylated POE ethers and alkyl polyglycosides such as lauryl glycoside, decyl glycoside and coconut glycoside.

Further advantageous anionic surfactants are

taurates, for example sodium lauroyl taurate and sodium methyl cocoyl taurate,

ether carboxylic acids, for example sodium laureth-13 carboxylate and sodium PEG-6 cocamide carboxylate,

phosphoric acid esters and salts, such as, for example, DEA-oleth-10 phosphate and dilaureth-4 phosphate.

alkylsulfonates, for example sodium coconut monoglyceride sulfate, sodium C₁₂₋₁₄ olefin sulfonate, sodium lauryl sulfoacetate and magnesium PEG-3 cocamide sulfate.

Further advantageous amphoteric surfactants are

N-alkylamino acids, for example aminopropylalkylglutamide, alkylamino-propionic acid, sodium alkylimidodipropionate and lauroamphocarboxy-glycinate.

Further advantageous nonionic surfactants are alcohols.

Further suitable anionic surfactants within the meaning of the invention are furthermore

acyl glutamates such as di-TEA-palmitoyl aspartate and sodium caprylic/capric glutamate,

acyl peptides, for example palmitoyl hydrolyzed milk protein, sodium cocoyl hydrolyzed soya protein and sodium/potassium cocoyl hydrolyzed collagen

and carboxylic acids and derivatives, such as

for example lauric acid, aluminum stearate, magnesium alkanolate and zinc undecylenate,

ester carboxylic acids, for example calcium stearoyl lactylate, laureth-6 citrate and sodium PEG-4 lauramide carboxylate,

alkylaryl sulfonates.

Other suitable cationic surfactants within the meaning of the present invention are furthermore

alkylamines,

alkylimidazoles and

ethoxylated amines.

Other suitable nonionic surfactants within the meaning of the present invention are furthermore amine oxides, such as cocamidopropylamine oxide.

It is advantageous within the meaning of the present invention if the content of one or more detergent surfactants in the cosmetic or dermatological cleansing emulsion is chosen from the range from 5 to 25% by weight, very particularly advantageously from 10 to 20% by weight, in each case based on the total weight of the preparations.

The oil phase of the cosmetic or dermatological cleansing emulsions within the meaning of the present invention is advantageously chosen from the group of the esters of saturated and/or unsaturated, branched and/or unbranched alkane-carboxylic acids of a chain length of 3 to 30 C atoms and saturated and/or unsaturated, branched and/or unbranched alcohols of a chain length of 3 to 30 C atoms, from the group of the esters of aromatic carboxylic acids and saturated and/or unsaturated, branched and/or unbranched alcohols of a chain length of 3 to 30 C atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semisynthetic and natural mixtures of such esters, e.g. jojoba oil.

Furthermore, the oil phase can advantageously be chosen from the group consisting of the branched and unbranched hydrocarbons and hydrocarbon waxes, of the silicone oils, of the dialkyl ethers, of the group consisting of the saturated or unsaturated, branched or unbranched alcohols, and of the fatty acid triglycerides, especially the triglycerol esters of saturated and/or unsaturated, branched and/or unbranched alkanecarboxylic acids of a chain length of 8 to 24, in particular 12 to 18, C atoms. The fatty acid triglycerides can, for example, advantageously be chosen from the group consisting of the synthetic, semisynthetic and natural oils, e.g. olive oil, sunflower oil, soybean oil, groundnut oil, rape seed oil, almond oil, palm oil, coconut oil, palm kernel oil and suchlike.

Any desired mixtures of such oil and wax components are also advantageously to be employed within the meaning of the present invention. It can optionally also be advantageous to employ waxes, for example cetyl palmitate, as the sole lipid component of the oil phase.

Advantageously, the oil phase is chosen from the group consisting of 2-ethylhexyl iso-stearate, octyldodecanol, isotridecyl isononanoate, isoeicosane, 2-ethylhexyl cocoate, C₁₂₋₁₅ alkyl benzoate, caprylic/capric acid triglyceride, dicaprylyl ether.

Mixtures of C₁₂₋₁₅ alkyl benzoate and 2-ethyl hexyl isostearate, mixtures of C₁₂₋₁₅ alkyl benzoate and isotridecyl isononanoate, and mixtures of C₁₂₋₁₅ alkyl benzoate, 2-ethyl-hexyl isostearate and isotridecyl isononanoate are particularly advantageous.

Of the hydrocarbons, paraffin oil, squalane and squalene are to be used advantageously within the meaning of the present invention.

Advantageously, the oil phase can furthermore contain cyclic or linear silicone oils or consist completely of such oils, where, however, it is preferred to use an additional content of other oil-phase components apart from the silicon oil or the silicon oils.

Advantageously, cyclomethicone (octamethylcyclotetrasiloxane) is employed as the silicone oil to be used according to the invention. However, other silicone oils are also to be used advantageously within the meaning of the present invention, for example hexamethylcyclotrisiloxane, polydimethylsiloxane, poly(methylphenyl-siloxane).

Mixtures of cyclomethicone and isotridecyl isononanoate, of cyclomethicone and 2-ethylhexyl isostearate are furthermore particularly advantageous.

The oil phase is furthermore advantageously chosen from the group consisting of the phospholipids. The phospholipids are phosphoric acid esters of acylated glycerols. Of very great importance among the phosphatidylcholines are, for example, the lecithins, which are distinguished by the general structure

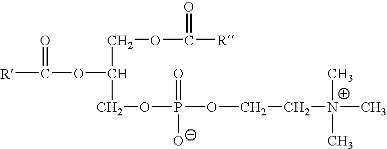

where R′ and R″ are typically unbranched aliphatic radicals having 15 or 17 carbon atoms and up to 4 cis double bonds.

Advantageous polyacrylates according to the invention are polymers of acrylic acid, in particular those which are chosen from the group consisting of the “carbomers” or “carbopols” (Carbopol® is actually a registered trademark of the B. F. Goodrich Company). Polyacrylates are compounds of the general structural formula

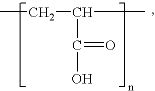

whose molecular weight can be between about 400 000 and more than 4 000 000. The group consisting of the polyacrylates furthermore includes acrylate/alkyl acrylate copolymers, for example those which are distinguished by the following structure:

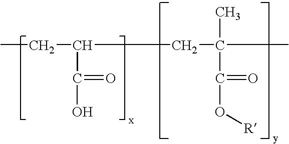

In this, R′ is a long-chain alkyl radical and x and y are numbers which symbolize the respective stoichiometric proportion of the respective comonomers. These polyacrylates are also advantageous within the meaning of the present invention.

Advantageous carbopols are, for example, the types 907, 910, 934, 940, 941, 951, 954, 980, 981, 1342, 1382, 2984 and 5984 or alternatively the types ETD (Easy-to-disperse) 2001, 2020, 2050, where these compounds can be present individually or in any desired combinations with one another.

Carbopol 981, 1382 and ETD 2020 (both individually and in combination) are particularly preferred.

The copolymers of C₁₀₋₃₀-alkyl acrylates and one or more monomers of acrylic acid, methacrylic acid or their esters comparable to the acrylate/alkyl acrylate copolymers are furthermore advantageous within the meaning of the present invention. The INCl designation for such compounds is “acrylates/C 10-30 alkyl acrylate crosspolymer”. Those obtainable under the trade names Pemulen TR1 and Pemulen TR2 from the B. F. Goodrich Company are particularly advantageous.

It is advantageous within the meaning of the present invention if the content of one or more polyacrylates in the cosmetic or dermatological cleansing emulsion is chosen from the range from 0.5 to 2% by weight, very particularly advantageously from 0.7 to 1.5% by weight, in each case based on the total weight of the preparations.

A novel particularly preferred embodiment of the invention are cleansing emulsions which, as detergent surfactants, contain at least one anionic surfactant and at least one thickener based on C₁₀-C₃₀-alkyl acrylates as polyacrylate. Sodium laureth sulfate is in this case particularly preferred as the anionic surfactant. This combination of ingredients is distinguished by its stability, its foam formation behavior, and by its particularly pleasant skin sensation.

Cosmetic preparations which are cosmetic cleansing preparations for the skin can be present in liquid or solid form.

The compositions contain, according to the invention, apart from the abovementioned substances, optionally the additives customary in cosmetics, for example perfume, colorants, antimicrobial substances, refatting agents, complexing and sequestering agents, pearl lustre agents, plant extracts, vitamins, active ingredients, preservatives, bactericides, pigments which have a coloring action, thickening agents, plasticizing, moistening and/or humectant substances, or other customary constituents of a cosmetic or dermatological formulation such as alcohols, polyols, polymers, foam stabilizers, electrolytes, organic solvents or silicone derivatives.

An additional content of antioxidants is in general preferred. According to the invention, favorable antioxidants which can be used are all antioxidants which are suitable or customary for cosmetic and/or dermatological applications.

Advantageously, the antioxidants are chosen from the group consisting of amino acids (e.g. glycine, histidine, tyrosine, tryptophan) and their derivatives, imidazoles (e.g. urocaninic acid) and their derivatives, peptides such as D,L-carnosine, D-carnosine, L-carnosine and their derivatives (e.g. anserine), carotenoids, carotenes (e.g. α-carotene, β-carotene, ψ-lycopene) and their derivatives, chlorogenic acid and its derivatives, lipoic acid and its derivatives (e.g. dihydrolipoic acid), aurothioglucose, propylthiouracil and other thiols (e.g. thioredoxin, glutathione, cysteine, cystine, cystamine and their glycosyl, N-acetyl, methyl, ethyl, propyl, amyl, butyl and lauryl, palmitoyl, oleyl, γ-linoleyl, cholesteryl and glyceryl esters), and their salts, dilauryl thiodipropionate, distearyl thiodipropionate, thiodipropionic acid and its derivatives (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerable doses (e.g. pmol to μmol/kg), furthermore (metal) chelators (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and their derivatives, unsaturated fatty acids and their derivatives (e.g. γ-linolenic acid, linoleic acid, oleic acid), folic acid and its derivatives, furfurylidenesorbitol and its derivatives, ubiquinone and ubiquinol and their derivatives, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of benzoin resin, rutic acid and its derivatives, α-glycosylrutin, ferulic acid, furfurylideneglucitol, carnosine, butylhydroxytoluene, butylhydroxy-anisole, nordihydroguaiaretic acid, nordihydroguaiaretic acid, trihydroxy-butyrophenone, uric acid and its derivatives, mannose and its derivatives, zinc and its derivatives (e.g. ZnO, ZnSO₄), selenium and its derivatives (e.g. selenomethionine), stilbenes and their derivatives (e.g. stilbene oxide, trans-stilbene oxide) and the derivatives suitable according to the invention (salts, esters, ethers, sugars, nucleotides, nucleosides, peptides and lipids) of these said active compounds.

The amount of the abovementioned antioxidants (one or more compounds) in the emulsions is preferably 0.001 to 30% by weight, particularly preferably 0.05 to 20% by weight, in particular 0.1-10% by weight, based on the total weight of the preparation.

If vitamin E and/or its derivatives is/are the antioxidant(s), their respective concentrations is advantageously to be chosen from the range from 0.001-10% by weight, based on the total weight of the formulation.

If vitamin A or vitamin A derivatives, or carotenes or their derivatives is/are the antioxidant(s), it is advantageous to choose their respective concentrations from the range from 0.001 to 10% by weight, based on the total weight of the formulation.

Surprisingly, it has now been found that all sorts of active ingredients having differing solubility can be incorporated homogeneously into the cleansing emulsions according to the invention. The substantivity of the active ingredients on skin and hair is significantly higher from the cleansing emulsion described than from conventional surfactant-containing cleansing formulations. It is to be suspected that the washing out of the active compounds from the skin by the surfactants contained in the formula is decreased or at least lowered by the formation of an oil film on the skin, such that a larger amount of the active ingredients contained in the product remains on the skin.

According to the invention, the active ingredients (one or more compounds) can very advantageously be chosen from the group consisting of the active ingredients, in particular from the following group:

acetylsalicylic acid, atropine, azulene, hydrocortisone and its derivatives, e.g. hydrocortisone 17-valerate, vitamins of the B and D series, very favorably vitamin B₁, vitamin B₁₂, vitamin D₁, vitamin A or its derivatives such as retinyl palmitate, vitamin E or its derivatives such as, for example, tocopheryl acetate, vitamin C and its derivatives such as, for example, ascorbyl glucuside but also bisabolol, unsaturated fatty acids, especially the essential fatty acids (often also called vitamin F), in particular γ-linolenic acid, oleic acid, eicosapentaenoic acid, docosahexaenoic acid and their derivatives, chloramphenicol, caffeine, prostaglandins, thymol, camphor, squalene, extracts or other products of vegetable and animal origin, e.g. evening primrose oil, borage oil or currant pip oil, fish oils, cod liver oil but also ceramides and ceramide-like compounds, frankincense extract, green tea extract, water lily extract, liquorice extract, Hamamelis.

It is also an advantage to choose the active ingredients from the group consisting of the refatting substances, for example purcellin oil, Eucerit® and Neocerit®.

Particularly advantageously, the active ingredients(s) is/are further chosen from the group consisting of the NO synthase inhibitors, in particular if the preparations according to the invention are intended to be used for the treatment and prophylaxis of the symptoms of intrinsic and/or extrinsic skin ageing, and for the treatment and prophylaxis of the harmful effects of ultraviolet radiation on the skin.

A preferred NO synthase inhibitor is nitroarginine.

Additionally advantageously, the active ingredient(s) is/are chosen from the group which includes catechols and bile esters of catechols and aqueous or organic extracts of plants or parts of plants which contain catechols or bile acid esters of catechols, such as, for example, the leaves of the plant family Theaceae, in particular of the species Camellia sinensis (green tea). Their typical ingredients (such as, for example, polyphenols or catechols, caffeine, vitamins, sugars, minerals, amino acids, lipids) are particularly advantageous.

Catechols are a group of compounds which are to be interpreted as hydrogenated flavones or anthocyanidins and derivatives of “catechol” (3,3′,4′,5,7-flavanpentaol, 2-(3,4-dihydroxyphenyl)chroman-3,5,7-triol). Epicatechol ((2R,3R)-3,3′,4′,5,7-flavan-pentaol) is also an advantageous active ingredient within the meaning of the present invention.

Plant extracts containing catechols are furthermore advantageous, in particular extracts of green tea, such as, for example, extracts of leaves of the plants of the species Camellia spec., very particularly the tea strains Camellia sinenis, C. assamica, C. taliensis or C. irrawadiensis and crossings of these with, for example, Camellia japonica.

Preferred active ingredients are further polyphenols or catechols from the group consisting of (−)-catechol, (+)-catechol, (−)-catechol gallate, (−)-gallocatechol gallate, (+)-epi-catechol, (−)-epicatechol, (−)-epicatechol gallate, (−)-epigallocatechol, (−)-epigallo-catechol gallate.

Flavone and its derivatives (often also collectively called “flavones”) are also advantageous active ingredients within the meaning of the present invention. They are characterized by the following basic structure (substitution positions indicated):

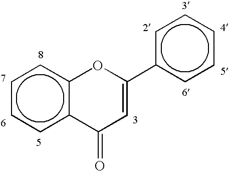

Some of the more important flavones, which can also preferably be employed in the preparations according to the invention, are listed in table 2 below:

TABLE 2

OH substitution positions

| | 3 | 5 | 7 | 8 | 2′ | 3′ | 4′ | 5′ |
|---|---|---|---|---|---|---|---|---|
| Flavone | − | − | − | − | − | − | − | − |
| Flavonol | + | − | − | − | − | − | − | − |
| Chrysin | − | + | + | − | − | − | − | − |
| Galangin | + | + | + | − | − | − | − | − |
| Apigenin | − | + | + | − | − | − | + | − |
| Fisetin | + | − | + | − | − | + | + | − |
| Luteolin | − | + | + | − | − | + | + | − |
| Kaempferol | + | + | + | − | − | − | + | − |
| Quercetin | + | + | + | − | − | + | + | − |
| Morin | + | + | + | − | + | − | + | − |
| Robinetin | + | − | + | − | − | + | + | + |
| Gossypetin | + | + | + | + | − | + | + | − |
| Myricetin | + | + | + | − | − | + | + | + |

In nature, flavones as a rule occur in glycosylated form.

According to the invention, the flavones are preferably chosen from the group consisting of the substances of the generic structural formula

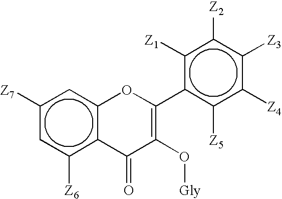

where Z₁ to Z₇ independently of one another are chosen from the group consisting of H, OH, alkoxy and hydroxyalkoxy groups, where the alkoxy or hydroxyalkoxy groups can be branched or unbranched and can have 1 to 18 C atoms, and where Gly is chosen from the group consisting of the mono- and oligoglycoside radicals.

According to the invention, the flavonoids, however, can also advantageously be chosen from the group consisting of the substances of the generic structural formula

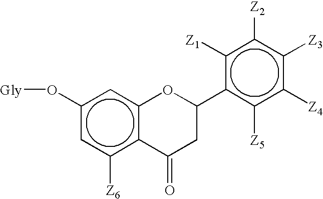

where Z₁ to Z₆ independently of one another are chosen from the group consisting of H, OH, alkoxy and hydroxyalkoxy groups, where the alkoxy or hydroxyalkoxy groups can be branched or unbranched and can have 1 to 18 C atoms, and where Gly is chosen from the group consisting of the mono- and oligoglycoside radicals.

Preferably, such structures can be chosen from the group consisting of the substances of the generic structural formula

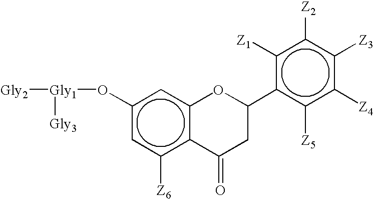

where Gly₁, Gly₂ and Gly₃ independently of one another are monoglycoside radicals or. Gly₂ and Gly₃ can also individually or together be saturations by hydrogen atoms.

Preferably, Gly₁, Gly₂ and Gly₃ independently of one another are chosen from the group consisting of the hexosyl radicals, in particular the rhamnosyl radicals and glucosyl radicals. However, other hexosyl radicals, for example allosyl, altrosyl, galactosyl, gulosyl, idosyl, mannosyl and talosyl are optionally also to be used advantageously. It can also be advantageous according to the invention to use pentosyl radicals.

Advantageously, Z₁ to Z₅ independently of one another are chosen from the group consisting of H, OH, methoxy, ethoxy and 2-hydroxyethoxy groups, and the flavone glycosides have the structure

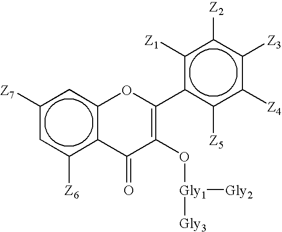

Particularly advantageously, the flavone glycosides according to the invention are from the group which are represented by the following structure:

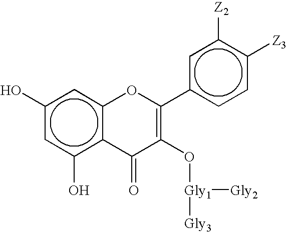

where Gly₁, Gly₂ and Gly₃ independently of one another are monoglycoside radicals. Gly₂ and Gly₃ can also individually or together be saturations by hydrogen atoms.

Preferably, Gly₁, Gly₂ and Gly₃ independently of one another are chosen from the group consisting of the hexosyl radicals, in particular the rhamnosyl radicals and glucosyl radicals. However, other hexosyl radicals, for example allosyl, altrosyl, galactosyl, gulosyl, idosyl, mannosyl and talosyl are optionally also to be used advantageously. It can also be advantageous according to the invention to use pentosyl radicals.

It is particularly advantageous within the meaning of the present invention to choose the flavone glycoside(s) from the group consisting of α-glucosylrutin, α-glucosyl-myricetin, α-glucosylisoquercitrin, α-glucosylisoquercetin and α-glucosylquercitrin.

According to the invention, α-glucosylrutin is particularly preferred.

According to the invention, naringin (aurantiin, naringenin 7-rhamnoglucoside), hesperidin (3′,5,7-trihydroxy-4′-methoxyflavanone 7-rutinoside, hesperidoside, hesperetin-7-O-rutinoside) are also advantageous. Rutin (3,3′,4′,5,7-pentahydroxyflyvone 3-rutinoside, quercetin 3-rutinoside, sophorin, birutan, rutabion, taurutin, phytomelin, melin), troxerutin (3,5-dihydroxy-3′,4′,7-tris(2-hydroxyethoxy)flavone 3-(6-O-(6-deoxy-α-L-mannopyranosyl)-β-D-glucopyranoside)), monoxerutin (3,3′,4′, 5-tetrahydroxy-7-(2-hydroxyethoxy)flavone 3-(6-O-(6-deoxy-α-L-mannopyranosyl)-β-D-glucopyranoside)), dihydrorobinetin (3,3′,4′,5′,7-pentahydroxy-flavanone), taxifolin (3,3′,4′,5,7-pentahydroxyflavanone), eriodictyol-7-glucoside (3′,4′,5,7-tetrahydroxyflavanone 7-glucoside), flavanomarein (3′,4′,7,8-tetrahydroxy-flavanone 7-glucoside) and isoquercetin (3,3′,4′,5,7-pentahydroxyflavanone 3-(β-D-glucopyranoside).

It is also advantageous to choose the active ingredient(s) from the group consisting of the ubiquinones and plastoquinones.

Ubiquinones are distinguished by the structural formula

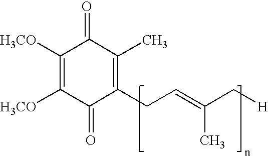

and are the most widespread and thus the best investigated bioquinones. Depending on the number of isoprene units linked in the side chain, ubiquinones are designated as Q-1, Q-2, Q-3 etc or on the number of C atoms as U-5, U-10, U-15 etc. They preferably occur with certain chain lengths, for example in some microorganisms and yeasts with n=6. In most mammals including man Q10 predominates.

Particularly advantageous is coenzyme Q10, which is characterized by the following structural formula:

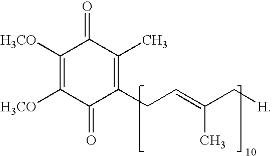

Plastoquinones have the general structural formula

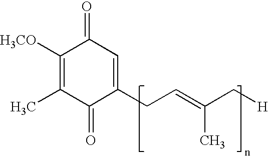

Plastoquinones differ in the number n of isoprene radicals and are designated accordingly, for example PQ-9 (n=9). Furthermore, other plastoquinones having different substituents on the quinone ring exist.

Creatine and/or creatine derivatives are also preferred active ingredients within the meaning of the present invention. Creatine is distinguished by the following structure:

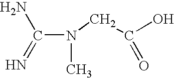

Preferred derivatives are creatine phosphate and creatine sulfate, creatine acetate, creatine ascorbate and the derivatives esterified on the carboxyl group by mono- or polyfunctional alcohols.

A further advantageous active ingredient is L-carnitine [3-hydroxy-4-(trimethylammonio)butyric acid betaine]. Acylcarnitines, which chosen from the group of substances of the following general structural formula

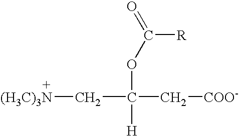

where R is chosen from the group consisting of the branched and unbranched alkyl radicals having up to 10 carbon atoms are also advantageous active ingredients within the meaning of the present invention. Propionylcarnitine and in particular acetylcarnitine are preferred. Both enantiomers (D- and L-form) can be used advantageously within the meaning of the present invention. It can also be advantageous to use any desired mixtures of enantiomers, for example a racemate of the D- and L-form.

Further advantageous active ingredients are sericoside, pyridoxol, vitamin K, biotin and aromatic substances.

Moreover, the active ingredients according to the invention (one or more compounds) can also very advantageously be chosen from the group consisting of the hydrophilic active ingredients, in particular from the following group: alpha hydroxy acids such as lactic acid or salicylic acid and their salts such as, for example, Na lactate, Ca lactate, TEA lactate, urea, allantoin, serine, sorbitol, glycerol, milk proteins, panthenol, chitosan.

The list of the active ingredients or active ingredient combinations mentioned, which can be used in the preparations according to the invention, should of course not be limiting. The active ingredients can be used individually or in any desired combinations with one another.

The amount of such active ingredients (one or more compounds) in the preparations according to the invention is preferably 0.001 to 30% by weight, particularly preferably 0.05-20% by weight, in particular 1-10% by weight, based on the total weight of the preparation.

The use of the cosmetic or dermatological cleansing emulsions as a foam, shower or tub bath, and as a hair shampoo, is novel.

The novel cosmetic or dermatological cleansing emulsions are advantageously used as a thickened, in particular cream-like, preparation.

Not least, the use of the cosmetic or dermatological cleansing emulsions for the prophylaxis and/or treatment of inflammatory skin conditions and/or for skin protection in the case of sensitively determined and dry skin is novel.

The following examples, in which washing preparations for hair and body care are described, are intended to illustrate the novel compositions without it being intended, however, to restrict the invention to these examples. The numerical values in the examples denote percentages by weight, based on the total weight of the respective

1.-12. (canceled).

13. A cosmetic or dermatological cleansing emulsion comprising:
(a) from 1% to 30% by weight of one or more detergent surfactants which have an HLB value of higher than 15;
(b) from 35% to 50% by weight of one or more oil components;
(c) from 0.001% to 30% by weight of one or more active ingredients;
(d) from 0.2% to 5% by weight of one or more polyacrylates selected from anionic homopolymers and anionic copolymers of at least one of acrylic acid, alkylated acrylic acid and esters thereof; and
(e) from 5% to 60% by weight of water.

14. The emulsion of claim 13, wherein the one or more detergent surfactants have an HLB value of higher than 25.

15. The emulsion of claim 13, wherein the one or more detergent surfactants have an HLB value of higher than 35.

16. The emulsion of claim 13, wherein the one or more detergent surfactants comprise one or more anionic surfactants.

17. The emulsion of claim 16, wherein the one or more anionic surfactants comprise at least one of acylamino acids and salts thereof, sulfonic acids and salts thereof, and sulfuric acid esters.

18. The emulsion of claim 13, wherein the one or more detergent surfactants comprise one or more cationic surfactants.

19. The emulsion of claim 18, wherein the one or more cationic surfactants comprise at least one quaternary surfactant.

20. The emulsion of claim 13, wherein the one or more detergent surfactants comprise one or more amphoteric surfactants.

21. The emulsion of claim 20, wherein the one or more amphoteric surfactants comprise at least one acyl-/dialkylethylenediamine.

22. The emulsion of claim 13, wherein the one or more detergent surfactants comprise one or more nonionic surfactants.

23. The emulsion of claim 22, wherein the one or more nonionic surfactants comprise at least one of an alkanolamide, an ester formed by esterification of a carboxylic acid with at least one of ethylene oxide, glycerol and sorbitan, and an ether.

24. The emulsion of claim 13, wherein the emulsion comprises from 5% to 25% by weight of component (a).

25. The emulsion of claim 14, wherein the emulsion comprises from 10% to 20% by weight of component (a).

26. The emulsion of claim 13, wherein component (a) comprises sodium laureth sulfate.

27. The emulsion of claim 24, wherein component (a) comprises at least one of sodium acyl glutamate, myristoyl sarcosine, TEA lauroyl sarcosinate, sodium lauroyl sarcosinate and sodium cocoyl sarcosinate, sodium/ammonium cocoyl isethionate, dioctyl sodium sulfosuccinate, disodium laureth sulfosuccinate, disodium lauryl sulfosuccinate and disodium undecyleneamido MEA sulfosuccinate, sodium, ammonium, magnesium, MIPA, TIPA laureth sulfate, sodium myreth sulfate and sodium C₁₂₋₁₃ pareth sulfate, sodium, ammonium and TEA lauryl sulfate, benzalkonium chloride, alkylbetaine, alkylamidopropylbetaine and alkylamidopropylhydroxysultaine, sodium acyl-amphoacetate, disodium acylamphodipropionate, disodium alkylamphodiacetate, sodium acylamphohydroxypropylsulfonate, disodium acylamphodiacetate and sodium acylamphopropionate, cocamide MEA/DEA/MIPA, lauryl glucoside, decyl glycoside and coconut glycoside.

28. The emulsion of claim 13, wherein component (b) comprises at least one of 2-ethylhexyl isostearate, octyldodecanol, isotridecyl isononanoate, isoeicosane, 2-ethylhexyl cocoate, C₁₂₋₁₅ alkyl benzoate, caprylic/capric acid triglyceride, and dicaprylyl ether.

29. The emulsion of claim 14, wherein component (b) comprises at least one of a mixture of C₁₂₋₁₅ alkyl benzoate and 2-ethyl hexyl isostearate, a mixture of C₁₂₋₁₅ alkyl benzoate and isotridecyl isononanoate, and a mixture of C₁₂₋₁₅ alkyl benzoate, 2-ethyl-hexyl isostearate and isotridecyl isononanoate.

30. The emulsion of claim 13, wherein component (c) comprises at least one of a polyphenol, a catechol, a flavonoid, a ubiquinone, a plastoquinone, creatine and derivatives thereof, a carnitine, vitamins A to K, an alpha-hydroxy acid, urea, allantoin, serine, sorbitol, glycerol, nitroarginine, a milk protein, pantheol, chitosan, sericoside, pyridoxol, hydrocortisone and a derivative thereof, and an essential fatty acid.

31. The emulsion of claim 14, wherein component (c) comprises an NO synthase inhibitor.

32. The emulsion of claim 30, wherein the emulsion comprises from 0.05% to 20% by weight of component (c).

33. The emulsion of claim 13, wherein component (d) comprises a polymer of at least one of acrylic acid and esters thereof, acrylate/alkyl acrylate copolymers and combinations thereof.

34. The emulsion of claim 33, wherein the emulsion comprises from 0.5% to 2% by weight of component (d).

35. The emulsion of claim 13, wherein the emulsion comprises at least one anionic surfactant and at least one C₁₀-C₃₀ alkyl acrylate polymer.

36. A cosmetic or dermatological cleansing emulsion comprising:
(a) from 5% to 20% by weight of one or more detergent surfactants which have an HLB value of higher than 15;
(b) from 35% to 50% by weight of one or more oil components;
(c) from 0.05% to 20% by weight of one or more active ingredients;
(d) from 0.5% to 2% by weight of one or more polyacrylates selected from at least one of anionic homopolymers and anionic copolymers of at least one of acrylic acid, alkylated acrylic acid and esters thereof; and
(e) from 5% to 55% by weight of water.

37. The emulsion of claim 36, wherein the one or more detergent surfactants comprise at least one surfactant having an HLB value of higher than 25.

38. The emulsion of claim 37, wherein the one or more detergent surfactants comprise one or more anionic surfactants.

39. The emulsion of claim 37, wherein the emulsion comprises from 10% to 20% by weight of component (a).

40. The emulsion of claim 36, wherein component (a) comprises sodium laureth sulfate.

41. The emulsion of claim 36, wherein component (a) comprises at least one of sodium acyl glutamate, myristoyl sarcosine, TEA lauroyl sarcosinate, sodium lauroyl sarcosinate and sodium cocoyl sarcosinate, sodium/ammonium cocoyl isethionate, dioctyl sodium sulfosuccinate, disodium laureth sulfosuccinate, disodium lauryl sulfosuccinate and disodium undecyleneamido MEA sulfosuccinate, sodium, ammonium, magnesium, MIPA, TIPA laureth sulfate, sodium myreth sulfate and sodium C₁₂₋₁₃ pareth sulfate, sodium, ammonium and TEA lauryl sulfate, benzalkonium chloride, alkylbetaine, alkylamidopropylbetaine and alkylamidopropylhydroxysultaine, sodium acyl-amphoacetate, disodium acylamphodipropionate, disodium alkylamphodiacetate, sodium acylamphohydroxypropylsulfonate, disodium acylamphodiacetate and sodium acylamphopropionate, cocamide MEA/DEA/MIPA, lauryl glucoside, decyl glycoside and coconut glycoside.

42. The emulsion of claim 41, wherein component (c) comprises at least one of a polyphenol, a catechol, a flavonoid, a ubiquinone, a plastoquinone, creatine and derivatives thereof, a carnitine, vitamins A to K, an alpha-hydroxy acid, urea, allantoin, serine, sorbitol, glycerol, nitroarginine, a milk protein, pantheol, chitosan, sericoside, pyridoxol, hydrocortisone and a derivative thereof, and an essential fatty acid.

43. The emulsion of claim 42, wherein component (d) comprises a polymer of at least one of acrylic acid and esters thereof, acrylate/alkyl acrylate copolymers, and combinations thereof.

44. The emulsion of claim 36, wherein the emulsion comprises at least one anionic surfactant and at least one C₁₀-C₃₀ alkyl acrylate polymer.

45. A foam bath which comprises the emulsion of claim 13.

46. A shower bath which comprises the emulsion of claim 13.

47. A tub bath which comprises the emulsion of claim 13.

48. A hair shampoo which comprises the emulsion of claim 13.

49. A cream-like preparation which comprises the emulsion of claim 13.

50. A method for the treatment or prophylaxis of inflammatory skin conditions, wherein the method comprises applying to at least parts of the skin the emulsion of claim 13.

51. A method of protecting sensitive and dry skin, wherein the method comprises applying to at least parts of the skin the emulsion of claim 13.
